(19) United States
(12) Patent Application Publication
(10) Pub. No.: US 2021/0228646 A1
(43) Pub. Date: Jul. 29, 2021

(54) PERSONALIZED LEUKEMIA/LYMPHOMA THERAPEUTIC MODEL

(71) Applicant: The University of Chicago, Chicago, IL (US)

(72) Inventors: Y. Lynn Wang, Chicago, IL (US); Pin Lu, Chicago, IL (US)

(21) Appl. No.: 17/051,068

(22) PCT Filed: Apr. 30, 2019
(86) PCT No.: PCT/US2019/030013

§ 371 Date: Oct. 27, 2020

Related U.S. Application Data
(60) Provisional application No. 62/665,344, filed on May 1, 2018.

(51) Int. Cl. A61K 35/35; A61K 35/17; A61K 45/06; A61P 35/02

(52) U.S. Cl. CPC A61K 35/35; A61P 35/02; A61K 45/06; A61K 35/17

(57) ABSTRACT

The disclosure is directed to an ex vivo cell culture system and methods of using the cell culture system to identify potential therapeutic agents for the treatment of leukemia or lymphoma, such as chronic lymphocytic leukemia (CLL). The ex vivo culture system comprises (a) a first cell culture comprising bone marrow stromal cells (BMSC) which express one or more exogenous cell signaling molecules; (b) a second cell culture comprising leukemia or lymphoma cells isolated from a human; and optionally (c) one or more soluble cell signaling molecules.

### CROSS-REFERENCE TO RELATED APPLICATIONS

This application claims the benefit of U.S. Provisional Application No. 62/665,344, filed May 1, 2018, which is incorporated by reference herein in its entirety.

### FIELD

The disclosure is directed to an ex vivo cell culture system and methods of using the cell culture system to select potential therapeutic agents for the treatment of individual patients with leukemia or lymphoma, such as chronic lymphocytic leukemia (CLL), subtypes of non-Hodgkin lymphoma (NHL) (e.g., small lymphocytic lymphoma (SLL), follicular lymphoma (FL), marginal zone lymphoma (MZL), mantle cell lymphoma (MCL), and diffuse large B-cell lymphoma (DLBCL)), and subtypes of T-cell lymphomas (e.g., peripheral T-cell lymphoma and T-prolymphocytic lymphoma, etc.). These conditions are collectively referred to as lymphoma hereafter.

The ex vivo culture system comprises (a) a first cell culture comprising bone marrow stromal cells (BMSC) which express one or more exogenous cell signaling molecules, (b) a second cell culture comprising leukemia or lymphoma cells isolated from a patient, and optionally (c) one or more soluble cell signaling molecules.

### BACKGROUND OF THE INVENTION

Chronic lymphocytic leukemia (CLL) is the most common leukemia/lymphoma among the elderly population. CLL is a chronic lymphoid malignancy characterized by an accumulation of monoclonal mature B-cells in peripheral blood (PB), bone marrow (BM), and secondary lymphoid tissues. Historically, CLL has been considered a disease of defective apoptosis, since tumor cells isolated from the peripheral circulation are dormant and non-proliferating. More recently, however, cell proliferation has been recognized as playing an important role in CLL pathogenesis (Deaglio et al., Haematologica. 2009; 94: 752-6; Messmer et al., The Journal of clinical investigation. 2005; 115:755-64; Schmid et al., Histopathology. 1994; 24:445-51; Lampert et al., Hum Pathol. 1999; 30:648-54; Fabbri et al., Nat Rev Cancer. 2016; 16:145-62; and Gine et al., Haematologica. 2010; 95:1526-33; herein incorporated by reference in their entireties).

CLL, overall, is a heterogeneous disease with different clinical presentations, IGHV mutational status, cytogenetic features, and genomic profiles. Patients typically are treated with chemotherapy, immunotherapy using monoclonal anti-CD20 antibodies, or chemoimmunotherapy regimens. A variety of targeted therapies, including ibrutinib (BTK inhibitor) and venetoclax (BCL2 inhibitor), have recently been developed and are generating high response rates, revolutionizing CLL treatment. However, any given individual patient may be intolerant or refractory to a particular therapy, and patients who respond initially may relapse with dismal outcomes. Therapy selection for each patient currently is based on clinical factors such as age, comorbidities, and prior therapies, but the behavior of an individual patient's tumor cells is not taken into consideration. Individualized therapy is especially needed for CLL patients with high-risk profiles, such as chromosomal 17p deletion or complex cytogenetics. These patients experience a higher rate of disease progression on the newer therapy ibrutinib (Maddocks et al., JAMA oncology. 2015; 1:80-7; and Kadri et al., Blood advances. 2017; 1:715-277; herein incorporated by reference in their entireties). Thus, reliable models of leukemia and lymphoma are needed in order to tailor therapy for each individual patient.

Unfortunately, drug testing models for CLL are lacking. A cell line model has been developed from a case of aggressive CLL with biallelic loss of TP53, and a TCL1 adoptive transfer animal model has been utilized to evaluate experimental therapies (Herman et al., Clin Cancer Res. 2017; 23:2831-41; and Niemann et al., Clin Cancer Res Aug. 29, 2017 DOI: 10.1158/1078-0432.CCR-17-0650; herein incorporated by reference in their entireties). The heterogeneity of CLL is not recapitulated by these models, however. Immune-compromised NSG mice bearing patient-derived xenografts (PDx) have been generated to address the concern for individualization and have been adopted recently for preclinical drug evaluations (Herman et al., Leukemia. 2013; 27:2311-21; Matas-Cespedes et al., Clin Cancer Res. 2017; 23:1493-505; and Davies et al., Oncotarget. 2017; 8:44749-60; herein incorporated by reference in their entireties). However, several months are required for the engraftment to occur, making PDx not amenable as a front-line therapeutic model for patients who need immediate treatment. This model is also costly, technically demanding, and requires specialized facilities such as animal rooms, further limiting the use of PDx as a personalized therapeutic tool.

With the increasing appreciation and understanding of the tumor microenvironment, attempts have been made to recreate the tumor microenvironment in vitro and then evaluate drug-induced apoptosis under more physiological conditions. In the lymph node microenvironment, CLL interacts with T-cells and various other types of stromal cells (Caligaris-Cappio et al., Semin Cancer Biol. 2014; 24:43-8; and Herishanu et al., Hematol Oncol Clin North Am. 2013; 27:173-206; herein incorporated by reference in their entireties). The microenvironment also provides various stimuli, such as adhesion molecules, cytokines, chemokines, growth factors, and autologous antigens that promote tumor hallmark behaviors such as, for example, survival, adhesion, migration and proliferation. In addition, studies have shown that the tumor microenvironment protects CLL cells from spontaneous and drug-induced apoptosis (Janel et al., Stem Cells Dev. 2014; 23:2972-82; Kurtova et al., Blood. 2009; 114:4441-50; and Purroy et al., Oncotarget. 2015; 6:7632-4; herein incorporated by reference in their entireties). Protection by the microenvironment may also be responsible for the persistence of minimal residual disease (MRD), as well as shorter progression-free and overall survival in treated patients (Boucher et al., Clin Oncol. 2012; 30:980-8; herein incorporated by reference in its entirety). Co-culture with nurse-like cells of the peripheral blood (PB) or bone marrow stromal cells (BMSC) has been shown to increase CLL cell survival and migration; however, few of the reported systems are robust enough to support CLL proliferation and none allow for visualization and quantification of cell adhesion.

The inability of the current available models to measure all of the important tumor properties has resulted in faulty drug leads that fail in patient-based clinical trials. Inadequate representation of the human tissue environment during a preclinical screen can result in inaccurate predictions of effects of drug candidates. Thus, pharmaceutical companies are constantly searching for preclinical models that closely resemble original tissue for predicting clinical outcome. Currently, cancer patients are treated empirically with the standard care that works in the majority of the patients with the same type of cancer. However, each patient is different. Not all available drugs are equally effective for a given patient. Patient heterogeneity due to age, sex, race, genetic background and tumor heterogeneity necessitates personalized approach of treatment. A practical personalized therapeutic model for leukemia and lymphoma that is accurate, fast, easy, and economical is currently non-existent.

There remains a need for accurate models of leukemias and lymphomas, such as CLL, in order to identify effective therapeutic agents tailored to individual patients.

### BRIEF SUMMARY OF THE INVENTION

The disclosure provides an ex vivo cell culture system, which comprises (a) a first cell culture comprising bone marrow stromal cells (BMSC) which express one or more exogenous cell signaling molecules; (b) a second cell culture comprising leukemia or lymphoma cells isolated from a human; and optionally (c) one or more soluble cell signaling molecules.

The disclosure also provides a method of preparing the aforementioned ex vivo cell culture system, which comprises: (a) contacting (e.g., cloning, engineering, etc.) bone marrow stromal cells (BMSC) with a vector comprising at least one nucleic acid sequence encoding at least one exogenous cell signaling molecule; (b) culturing the BMSC under conditions whereby the at least one nucleic acid sequence is expressed and the at least exogenous one cell signaling molecule is produced; (c) contacting (e.g., treating) the BMSC with leukemia or lymphoma cells isolated from a human and optionally one or more soluble cell signaling molecules, and (d) culturing the BMSC and leukemia or lymphoma cells under suitable conditions whereby the ex vivo culture system is established.

The disclosure further provides a method of identifying an agent that inhibits leukemia or lymphoma, which comprises (a) treating the aforementioned ex vivo cell culture system with at least one candidate agent (e.g., a single agent or a combination of agents); (b) measuring one or more of survival, proliferation, adhesion, and/or migration of the leukemia or lymphoma cells following step (a), wherein a decrease in survival, proliferation, adhesion, and/or migration of the leukemia or lymphoma cells as compared to cells not treated with the at least one candidate agent indicates that the at least one candidate agent inhibits leukemia or lymphoma.

The disclosure also provides a method of treating leukemia or lymphoma in a subject in need thereof. The method comprises (a) isolating leukemia or lymphoma cells from the subject, (b) preparing an ex vivo cell culture system according to the methods described herein; (c) treating the ex vivo cell culture system with at least one candidate therapeutic agent; (d) measuring one or more of survival, proliferation, adhesion, and/or migration of the leukemia or lymphoma cells following step (c), wherein a decrease in survival, proliferation, adhesion, and/or migration of the leukemia or lymphoma cells as compared to cells not treated with the at least one candidate therapeutic agent indicates that the at least one candidate therapeutic agent inhibits the leukemia or lymphoma; and (e) administering the at least one candidate therapeutic agent to the subject, whereby the leukemia or lymphoma is treated.

The disclosure also provides a method of treating leukemia or lymphoma in a subject in need thereof. The method comprises (a) isolating leukemia or lymphoma cells from the subject, (b) determining whether at least one candidate agent inhibits leukemia or lymphoma by having the leukemia or lymphoma cells tested in an ex vivo cell culture system described herein; and (c) administering the at least one candidate therapeutic agent to the subject, whereby the leukemia or lymphoma is treated.

### BRIEF DESCRIPTION OF THE DRAWING(S)

FIGS. 1A-1C are graphs illustrating detection of daughter cell proliferation of CLL cells co-cultured with BMSC in the presence of soluble cell signaling molecules (IL-15 and CpG-oligodeoxynucleotides). FIG. 1A is a histogram of magnetic-bead isolated, CSFE-labeled human CD4+ T cells after 4 days of in vitro stimulation. FIG. 1B is a graph showing CSFE labeling of an isolated CLL cell culture, and FIG. 1C is a graph showing CSFE labeling of CLL cells co-cultured with BMSC cells in the presence of IL-15 and CpG.

FIGS. 2A-2F are graphs illustrating CLL cell proliferation in a co-culture of BMSC and patient-isolated CLL cells treated with ibrutinib (Irb). BMSC and CLL cells were co-cultured in the presence of IL-15 and CpG as described herein. FIGS. 2A and 2B show percentage of live cells in Ibr-sensitive CLL populations (FIG. 2A) and Ibr-resistant CLL populations (FIG. 2B). FIGS. 2C-2F show CSFE labeling of Ibr-sensitive (FIGS. 2C and 2D) and resistant CLL populations (FIGS. 2E and 2F).

FIGS. 3A-3D are graphs showing that the modeled CLL proliferation responses to Ibr correlate well with patients' clinical response. FIG. 3A shows live CLL cell numbers and FIG. 3B shows percentage of CLL cells in proliferation in Ibr-sensitive populations. FIG. 3C shows live CLL cell numbers and FIG. 3D shows percentage of CLL cells in proliferation in Ibr-relapsed populations. BMSC and patient-isolated CLL cells were co-cultured in the presence of IL-15 and CpG as described herein.

FIGS. 4A and 4B are fluorescent confocal microscopy images of the ex-vivo cell culture system described herein in which the BMSC express CD40L. The images show that the CLL tumor cells (green) are in close contact with the CD40L-expressing BMSC (red) at two different magnifications captured by con-focal microscopy (A&B).

FIGS. 5A-5D are light microscopy images of the T prolymphocytic lymphoma co-cultured with the following BMSC: Basic: BMSC not expressing any cell signaling molecule (FIG. 5A); BMSC-1: CD40L-expressing BMSC (FIG. 5B); BMSC-2: triple cytokine-expressing BMSC+CD40L-expressing BMSC (FIG. 5C); BMSC-3: Basic BMSC+triple cytokine-expressing BMSC+CD40L-expressing BMSC (FIG. 5D). The light microscopic images show clustering of T-lymphoma cells that are co-cultured with different types of molecular-engineered BMSC. The most prominent clustering is observed with BMSC-2 (FIG. 5C) FIGS. 5E and 5F are flow cytometric graphs illustrating proliferation of primary T cell lymphoma using CFSE staining. The proliferative cell populations display low levels of CFSE. FIG. 5F shows time-dependent prominent proliferation of T-lymphoma cells co-cultured with BMSC-2 as opposed to complete lack of proliferation under basic BMSC in FIG. 5E.

FIGS. 6A and 6B are images of primary diffuse large B-cell lymphoma co-cultured with “Basic” BMSC (FIG. 8A) and BMSC-2 (FIG. 8B). The images show that BMSC-2 promotes cell cluster formation, hence cell proliferation, more effectively than the basic BMSC. FIGS. 6C and 6D are flow cytometric graphs illustrating proliferation of primary diffuse large B-cell lymphoma (DLBCL) co-cultured with the following BMSC: Basic: BMSC not expressing any cell signaling molecule; BMSC-1′: triple cytokine-expressing BMSC; BMSC-1: CD40L-expressing BMSC; BMSC-2: triple cytokine-expressing BMSC+CD40L-expressing BMSC. FIG. 6A shows cell viabilities under these conditions. Again, by comparing different co-culture conditions in parallel, the graphs demonstrate that the BMSC-2 condition provides the best support for B-lymphoma cells to proliferate.

### DETAILED DESCRIPTION OF THE INVENTION

The present disclosure is predicated, at least in part, on the discovery that co-culturing bone marrow stromal cells (BMSC) engineered to express one or more cell signaling molecules (e.g., growth factors and/or cytokines) together with leukemia or lymphoma cells isolated from a human patient recapitulates the tumor microenvironment and provides a model to test for drug sensitivity in a wholistic way on a subject-specific basis.

In this regard, provided herein are ex vivo cell culture systems, which comprises (a) a first cell culture comprising bone marrow stromal cells (BMSC) which express one or more exogenous cell signaling molecules; (b) a second cell culture comprising leukemia or lymphoma cells isolated from a human; and optionally (c) one or more soluble cell signaling molecules. As used herein, the term “ex vivo” refers to methods conducted within or on cells or tissue taken from a human in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context, such as experiments conducted with cell lines

The terms “cell culture” and “culture” are used synonymously herein and refer to the process by which cells are removed from an animal or plant and their subsequent growth in a favorable artificial environment. Cell culture conditions vary depending on the cell type, but generally include a suitable vessel with a substrate or medium that supplies the essential nutrients (e.g., amino acids, carbohydrates, vitamins, minerals), growth factors, hormones, and gases (e.g., CO₂, O₂), and regulates the physio-chemical environment (e.g., pH buffer, osmotic pressure, temperature). Most cells require a surface or an artificial substrate (adherent or monolayer culture) whereas others can be grown free floating in culture medium (suspension culture). The cells that are removed from an animal or plant and then grown under artificial conditions also are referred to as a “cell culture” or “culture” of cells. Cell culture methods and systems that may be used in the context of the present disclosure are known in the art (see, e.g., Freshney, R. Ian (ed.), Culture of Animal Cells: A Manual of Basic Technique and Specialized Applications, 7^{th} ed., Wiley-Blackwell (2016); herein incorporated by reference in its entirety) and are available from a variety of commercial sources.

In some embodiments, the culture system comprises a first cell culture comprising bone marrow stromal cells (BMSC). Bone marrow stroma comprises a heterogeneous population of cells that provide the structural and physiological support for hematopoietic cells. Bone marrow stroma also contains cells with a stem-cell-like character that allows them to differentiate into bone, cartilage, adipocytes, and hematopoietic supporting tissues. In culture, BMSC can be separated from hematopoietic cells by their differential adhesion to tissue culture plastic and their prolonged proliferative potential. In cultures generated from single-cell suspensions of marrow, bone marrow stromal cells grow in colonies, each derived from a single precursor cell termed the colony-forming unit-fibroblast (see, e.g., Krebsbach et al., Crit. Rev. Oral Biol. Med., 10(2): 165-181 (1999); and Bianco et al., Stem Cells, 19: 180-192 (2001); herein incorporated by reference in their entireties). In certain embodiments, BMSCs are isolated and cultured using any suitable method known in the art, such as those described in, e.g., Ramakrishnan et al., Methods Mol., Biol., 1035: 75-101 (2013); and Nemeth et al., Curr Protoc Immunol., 102: Unit 22F.12 (2013); herein incorporated by reference in their entireties).

In some embodiments, the BMSCs of the first cell culture express one or more exogenous cell signaling molecules. In this regard, the BMSCs may be genetically engineered or otherwise modified to contain a non-native or exogenous nucleic acid sequence encoding a cell signaling molecule. The term “nucleic acid sequence,” as used herein, is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, for example, methylated and/or capped polynucleotides.

An “exogenous” or “non-native” nucleic acid sequence is any nucleic acid sequence (e.g., DNA, RNA, or cDNA sequence) that is not a naturally occurring nucleic acid sequence of a BMSC in a naturally occurring position. For example, the exogenous nucleic acid sequence can be naturally found in the genome of a BMSC, but located at a non-native position within the BMSC genome and/or operably linked to a non-native promoter. Preferably, an exogenous nucleic acid sequence is not naturally expressed by the genome of a BMSC. The terms “non-native nucleic acid sequence,” “heterologous nucleic acid sequence,” and “exogenous nucleic acid sequence” are synonymous and can be used interchangeably in the context of the disclosure. The non-native nucleic acid sequence preferably is DNA and preferably encodes a protein (i.e., one or more nucleic acid sequences encoding one or more proteins).

The one or more exogenous nucleic acid sequences encoding a cell signaling molecule may be introduced into a BMSC by “transfection,” or “transduction.” “Transfection,” or “transduction,” as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991); herein incorporated by reference in its entirety); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

In some embodiments, bone marrow stromal cells (BMSC) may be contacted (e.g., genetically engineered or cloned) with a vector comprising at least one exogenous nucleic acid sequence encoding at least one cell signaling molecule. The vector can be, for example, a plasmid, a cosmid, a viral vector (e.g., retroviral or adenoviral), or a phage. Suitable vectors and methods of vector preparation are well known in the art (see, e.g., Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994; herein incorporated by reference in its entirety).

In some embodiments, the vector is a lentivirus vector. Lentiviruses are a subclass of Retroviruses. The lentivirus genome is monopartite, linear, dimeric, positive-strand single-stranded RNA (“ssRNA (+)”) of 9.75 kb, with a 5′-cap and a 3′poly-A tail. The lentiviral genome is flanked by 5′ and 3′ long terminal repeat (LTR) sequences which have promoter/enhancer activity and are essential for the correct expression of the full-length lentiviral vector transcript. The LTRs also have an important role in reverse transcription and integration of the vector into the target cell genome. Upon viral entry into a cell, the RNA genome is reverse-transcribed into double-stranded DNA, which is then inserted into the genome at a random position by the viral integrase enzyme. The lentivirus, now called a provirus, remains in the genome and is passed on to the progeny of the cell when it divides. Species of lentivirus include, for example, human immunodeficiency virus 1 (HIV-1), human immunodeficiency virus 2 (HIV-2), simian immunodeficiency virus (SIV), bovine immunodeficiency virus (BIV), and feline immunodeficiency virus (FIV). The lentiviral vector can be based on any lentivirus species.

Lentiviral vectors typically are generated by trans-complementation in packaging cells that are co-transfected with a plasmid containing the vector genome and the packaging constructs that encode only the proteins essential for lentiviral assembly and function. A self-inactivating (SIN) lentiviral vector can be generated by abolishing the intrinsic promoter/enhancer activity of the HIV-1 LTR, which reduces the likelihood of aberrant expression of cellular coding sequences located adjacent to the vector integration site (see, e.g., Vigna et al., J. Gene Med., 2: 308-316 (2000); Naldini et al., Science, 272: 263-267 (1996); and Mátrai et al., Molecular Therapy, 18(3): 477-490 (2010); herein incorporated by reference in their entireties). The most common procedure to generate lentiviral vectors is to co-transfect cell lines (e.g., 293T human embryonic kidney cells) with a lentiviral vector plasmid and three packaging constructs encoding the viral Gag-Pol, Rev-Tat, and envelope (Env) proteins.

In certain embodiments, BMSCs are engineered to express one or more cell signaling molecules using methods other than gene transfer technology. For example, the BMSCs may be genetically engineered to express one or more cell signaling molecules using gene editing methodologies such as CRISPR (clustered regularly interspaced short palindromic repeat). The terms “CRISPR” or “CRISPR-Cas9,” as used herein, refer to the various CRISPR-Cas9 and -CPF1, (and other) systems that can be programmed to target specific stretches of a genome and to edit DNA at precise locations. CRISPR-Cas9 gene editing systems are based on the RNA-guided Cas9 nuclease from the type II prokaryotic clustered regularly interspaced short palindromic repeats (CRISPR) adaptive immune system (see, e.g., Jinek et al., Science, 337: 816 (2012); Gasiunas et al, Proc. Natl. Acad. Set U.S.A., 109, E2579 (2012); Garneau et al., Nature, 468: 67 (2010); Deveau et al., Annu. Rev. Microbiol, 64: 475 (2010); Horvath and Barrangou, Science, 327: 167 (2010); Makarova et al., Nat. Rev. Microbiol., 9, 467 (2011); Bhaya et al., Annu. Rev. Genet., 45, 273 (2011); and Cong et al., Science, 339: 819-823 (2013); herein incorporated by reference in their entireties). CRISPR gene editing systems have been developed to enable targeted modifications to a specific gene of interest in eukaryotic cells (see, e.g., Cong et al., supra; Xiao-Jie et al., J. Med. Genet., 52(5): 289-96 (2015); U.S. Pat. No. 8,697,359; Xie et al., Genome Res., 24(9): 1526-1533 (2014); Huang et al., Stem Cells, 33(5): 1470-1479 (2015); Smith et al., Molecular Therapy, 23(3): 570-577 (2015); and U.S. Patent Application Publication 2014/0068797; herein incorporated by reference in their entireties). Methods for utilizing CRISPR technology for gene editing are described in, for example, Barrangou et al., Science 315, 1709-1712 (2007); Bolotin et al., Microbiology, 151, 2551-2561 (2005); Brouns et al., Science 321, 960-964 (2008); Cong et al., supra; Deitcheva et al., Nature 471, 602-607 (2011); Gasiunas et al., supra; Hale et al., Cell 139, 945-956 (2009); Jinek et al., Science 337, 816-821 (2012); Makarova et al., Biology Direct 2006, 1:7 (2006); Mali et al., Science 339, 823-826 (2013); Marraffini et al., Science 322, 1843-1845 (2008); Mojica et al., J Mol Evol 60, 174-182 (2005); Pourcel et al., Microbiology 151, 653-663 (2005); and Sapranauskas et al., Nucl. Acids Res. 39, gkr606-gkr9282 (2011); herein incorporated by reference in their entireties.

The term “cell signaling molecule,” as used herein, refers to a substance that interacts with a target cell and initiates transmission of stimuli via a signaling cascade to effector molecules that orchestrate an appropriate response. A cell signaling molecule can transmit stimuli, for example, by acting as a ligand to cell surface receptors and/or by entering the cell through its membrane or endocytosis. The multiple varieties of signaling induced by signaling molecules are frequently divided into three general categories based on the distance over which signals are transmitted. In endocrine signaling, the signaling molecules (e.g., hormones) are secreted by specialized endocrine cells and carried through the circulation to act on target cells at distant body sites. In paracrine signaling, a molecule released by one cell acts on neighboring target cells (e.g., the action of neurotransmitters in carrying signals between nerve cells at a synapse). In autocrine signaling, cells respond to signaling molecules that they themselves produce (e.g., response of cells of the vertebrate immune system to foreign antigens).

The one or more cell signaling molecules may be a gas, a small molecule, a peptide, or a protein. The BMSC may express any type of cell signaling molecule. Various types of cell signaling molecules include, but are not limited to, steroid hormones and steroid receptor superfamily members (e.g., testosterone, estrogen, progesterone, corticosteroids, and ecdysone), nitric oxide, carbon monoxide, neurotransmitters (e.g., acetylcholine, dopamine, epinephrine (adrenaline), serotonin, histamine, glutamate, glycine, and γ-aminobutyric acid), peptide hormones (e.g., insulin, glucagon, growth hormone, follicle-stimulating hormone, and prolactin), growth factors (e.g., nerve growth factor (NGF), epidermal growth factor (EGF), and platelet derived growth factor (PDGF)), cytokines (e.g., interleukins, interferons, and CD40 ligand (CD40L)), chemokines (e.g., CCL21, CCL25, CCL27, CXCL12 and CXCL13), CpG-oligodeoxynucleotides, and ligands. Cell signaling molecules are described in more detail in, for example, Cooper, G. M., The Cell: A Molecular Approach, 2^{nd} Ed., Sunderland (MA): Sinauer Associates (2000). Cytokines and growth factors are described in more detail in, for example, Dinarello, Charles A. “Historical Review of Cytokines.” European journal of immunology 37.Suppl 1 (2007): S34-S45 PMC. Web. 26 Apr. 2018; Turner et al., Biochimia Biophysica Acta—Molecular Cell Research, 1843(11): 2563-2582 (2014); and James, R. and R. A. Bradshaw, Ann. Rev. Biochem., 53(1): 259-292 (1984). In some embodiments, the exogenous cell signaling molecule is an interleukin, a chemokine, a TNF protein superfamily member, a CpG deoxyoligonucleotide, or a combination of any of the foregoing. For example, the exogenous cell signaling molecule may be interleukin-4 (IL-4), interleukin-15 (IL-15), interleukin-21 (IL-21), interleukin-6 (IL-6), interleukin-8 (IL-8), interleukin-10 (IL-10), CD40 ligand (CD40L), CpG deoxyoligonucleotides, chemokine (C-X-C motif) ligand 12 (CXCL12), chemokine (C-X-C motif) ligand 13 (CXCL13), B-cell activating factor (BAFF), and/or a proliferation inducing ligand (APRIL).

In some embodiments, the BMSC express a single exogenous cell signaling molecule. In other embodiments, the BMSC express more than one (e.g., 2, 3, 4, 5 or more) cell signaling molecules. In embodiments where the BMSC express multiple cell signaling molecules, the BMSC may comprise either a single nucleic acid sequence encoding the multiple cell signaling molecules, or multiple nucleic acid sequences each of which encodes a single cell signaling molecule.

In addition to the exogenous cell signaling molecule expressed by the BMSC of the first cell culture, the ex vivo culture system disclosed herein may optionally comprise one or more soluble cell signaling molecules. The term “soluble,” as used herein, is used herein to describe a cell signaling molecule that is not encoded by or otherwise expressed by a cell, but rather is provided in the cell culture medium. The one or more soluble cell signaling molecules may be any of the cell signaling molecules described above, or otherwise known in the art. In some embodiments, the one or more soluble cell signaling molecules may be a CpG deoxyoligonucleotide and/or IL-15. The one or more soluble cell signaling molecules may stimulate the proliferation and differentiation of the BMSC.

In addition to the first cell culture comprising BMSC, the ex vivo culture system described herein further comprises a second cell culture comprising leukemia or lymphoma cells isolated from a human patient. The leukemia or lymphoma cells may be isolated from a human suffering from any type of leukemia or lymphoma. Leukemias are cancers that develop in the bone marrow and result in high numbers of white blood cells (WBC). The four primary types of leukemia include acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL). Lymphomas are blood cancers that develop from lymphocytes and are typically classified as Hodgkin or non-Hodgkin's lymphoma. Non-Hodgkin's lymphomas include, but are not limited to, B cell lymphomas (e.g., diffuse large B-cell lymphoma (DLBCL)), follicular lymphoma, small lymphocytic lymphoma (SLL), mantle cell lymphoma (MCL), marginal zone lymphomas, Burkitt lymphoma, lymphoplasmacytic lymphoma, and T cell lymphomas (e.g., peripheral T-cell lymphomas and T-prolymphocytic lymphoma). In some embodiments, the second cell culture comprises leukemia cells, desirably chronic lymphocytic leukemia (CLL) cells.

Leukemia or lymphoma cells, including CLL cells, may be isolated from a human using any suitable method known in the art, such as those described in, for example, Drexler H. G., “Isolation and Culture of Leukemia Cell Lines,” In: Langdon S. P. (eds), Cancer Cell Culture. Methods in Molecular Medicine, vol 88. Humana Press (2004); Kellner et al., Leukemia Res., 40, Pages 54-59 (2016); and Hayes et al., Leukemia Res., 34(6): 809-815 (2010); herein incorporated by reference in their entireties. Kits and systems for isolating leukemia and lymphoma cells (e.g., ROSETTESEP™ Human B Cell Enrichment Cocktail (STEMCELL Technologies, Vancouver, BC)) also are commercially available from a variety of sources and may be used in the methods described herein.

In some embodiments, provided herein are methods of preparing the ex vivo cell culture system described herein. The method comprises (a) contacting bone marrow stromal cells (BMSC) with a vector comprising at least one nucleic acid sequence encoding at least one exogenous cell signaling molecule; (b) culturing the BMSC under conditions whereby the at least one nucleic acid sequence is expressed and the at least one cell signaling molecule is produced; (c) contacting BMSC with leukemia or lymphoma cells isolated from a human and optionally one or more soluble cell signaling molecules, and (d) culturing the BMSC and leukemia or lymphoma cells under suitable conditions whereby the ex vivo culture system is prepared and established. Descriptions of the BMSC, vector, exogenous and soluble cell signaling molecules, leukemia or lymphoma cells, and cell culture conditions described herein with respect to the ex vivo culture system also are applicable to those same aspects of the aforementioned method of preparing the ex vivo cell culture system.

In some embodiments, provided herein are methods of preparing an ex vivo cell culture system comprising (a) contacting a culture of bone marrow stromal cells (BMSC) expressing at least one exogenous cell signaling molecule with leukemia or lymphoma cells isolated from a human and optionally one or more soluble cell signaling molecules, and (b) culturing the BMSC and leukemia or lymphoma cells under suitable conditions whereby the ex vivo culture system is prepared. Descriptions of the BMSC, vector, exogenous cell signaling molecule, leukemia or lymphoma cells, and cell culture conditions described herein with respect to the ex vivo culture system also are applicable to those same aspects of the aforementioned method of preparing the ex vivo cell culture system.

In some embodiments, provided herein are methods of identifying and selecting an agent that inhibits leukemia or lymphoma of a particular patient, as each patient's tumor is different from the other. The methods comprise (a) treating the ex vivo cell culture system described herein with at least one candidate agent; (b) measuring one or more of survival, proliferation, adhesion, and/or migration of the leukemia or lymphoma cells following step (a), wherein a decrease in survival, proliferation, adhesion, and/or migration of the leukemia or lymphoma cells as compared to cells not treated with the at least one candidate agent indicates that the candidate agent inhibits the particular leukemia or lymphoma under testing. It will be appreciated that the response to the at least one candidate agent may vary from one patient to another.

The term “candidate agent,” as used herein, refers to any substance, compound, or molecule that may inhibit the initiation, promotion, or progression of a leukemia or lymphoma. In some embodiments, the candidate agent may be a small molecule (e.g., ibrutinib, idelalisib, and venetoclax), a chemotherapeutic agent (e.g., cyclophosphamide, hydroxydaunorubicin (doxorubicin), vincristine (Oncovin), and prednisone), a biologic agent (e.g., a monoclonal antibody), or an immunotherapeutic agent (e.g., CAR-T cell therapy). The term “inhibit,” as used herein, refers to the ability to substantially antagonize, prohibit, prevent, restrain, slow, disrupt, alter, eliminate, stop, or reverse the initiation, progression, or severity of, for example, a leukemia or a lymphoma. Thus, in the context of the present disclosure, a candidate agent “inhibits” leukemia or lymphoma if it promotes the inhibition of leukemia or lymphoma cell proliferation, the inhibition of vascularization of solid tumors (e.g., lymphoma), the eradication of leukemia. or lymphoma. cells, and/or a reduction in the size of at leak one tumor (e.g., a lymphoma). In some embodiments, the ex vivo cell culture is treated with at least one candidate agent. In other embodiments, the ex vivo cell culture is treated with more than one candidate agent (e.g., 2, 3, 4, or 5 or more candidate agents).

In some embodiments, the at least one candidate agent decreases survival, proliferation, adhesion, and/or migration of the leukemia or lymphoma cells as compared to cells not contacted with the candidate agent (i.e., control cells). The ability of a candidate agent to affect survival, proliferation, adhesion, and/or migration of the leukemia or lymphoma cells in the culture system can be determined using suitable methods known in the art. For example, cell survival may be measured using Annexin V/PI staining assay (e.g., Dead Cell Apoptosis Kit with Annexin V FITC and PI, ThermoFisher Scientific, Waltham, Mass.), cell division may be measured using a carboxyfluorescein succinimidyl ester (CFSE)-staining assay (e.g., CELLTRACE™ Violet Cell Proliferation Kit, ThermoFisher Scientific, Waltham, Mass.), cell proliferation may be assessed by Ki67 staining, adhesion between leukemia or lymphoma cells and the BMSCs may be assessed using a modified cell adhesion assay (see, e.g., de Rooij et al., Blood. 2012; 119:2590-4; and Herman et al., Clin Cancer Res. 2015; 21:4642-51; herein incorporated by reference in its entirety) and/or by measuring the cell surface expression of adhesion molecules, such as CD11c, CD44, CD49d, CD54, and CXCR4. Interaction between the leukemia or lymphoma cells and BMSC may be visualized using confocal microscopy. Cell migration may be assessed using a chemotaxis migration assay (see, e.g., Purroy et al., Oncotarget. 2017; 8:742-56; herein incorporated by reference in its entirety), and a pseudoemperipolesis assay may be used to measure leukemia or lymphoma cells migrating beneath the stromal monolayer (see, e.g., Chang et al., Blood. 2013; 122:2412-24; and Burger et al., Blood. 1999; 94:3658-67; herein incorporated by reference in their entireties). Cell size may be assessed using flow cytometry- or microscopy-based assays.

Due to the heterogeneity of certain leukemias and lymphomas, the ability of and degree to which a candidate agent may decrease survival, proliferation, adhesion, and/or migration of leukemia or lymphoma cells may vary from patient to patient. As such, the ability of a candidate agent to inhibit leukemia or lymphoma in accordance with the present disclosure is subject-specific. Thus, the methods described herein allow for the identification of therapeutic agents that are targeted to a particular patient based on the clinical presentation, composition, and genetic makeup of the individual leukemia or lymphoma. Accordingly, the disclosure also provides a method of treating leukemia or lymphoma in a subject in need thereof. In some embodiments, methods comprise (a) isolating leukemia or lymphoma cells from the subject, (b) preparing an ex vivo cell culture system using the methods described herein; (c) contacting the ex vivo cell culture system with a candidate therapeutic agent; (d) measuring one or more of survival, proliferation, adhesion, and/or migration of the leukemia or lymphoma cells following step (c), wherein a decrease in survival, proliferation, adhesion, and/or migration of the leukemia or lymphoma cells as compared to cells not contacted with the candidate therapeutic agent indicates that the candidate therapeutic agent inhibits the leukemia or lymphoma; and (e) administering the candidate therapeutic agent to the subject, whereby the leukemia or lymphoma is treated. In some embodiments, methods comprise (a) isolating leukemia or lymphoma cells from the subject, (b) contacting an ex vivo cell culture system comprising BMSCs expressing an exogenous signaling molecule with the leukemia or lymphoma cells and optionally one or more soluble cell signaling molecules; (c) treating the ex vivo cell culture system with at least one candidate therapeutic agent; (d) measuring one or more of survival, proliferation, adhesion, and/or migration of the leukemia or lymphoma cells, wherein a decrease in survival, proliferation, adhesion, and/or migration of the leukemia or lymphoma cells as compared to cells not treated with the at least one candidate therapeutic agent indicates that the at least one candidate therapeutic agent inhibits the leukemia or lymphoma; and (e) administering the at least one candidate therapeutic agent to the subject, whereby the leukemia or lymphoma is treated. In some embodiments, methods comprise (a) isolating leukemia or lymphoma cells from the subject, (b) testing whether the at least one candidate therapeutic agent inhibits the leukemia or lymphoma cells of the subject by having the cells tested in an ex vivo cell culture system described herein; and (c) administering the at least one candidate therapeutic agent to the subject, whereby the leukemia or lymphoma is treated. Descriptions of the BMSC, leukemia or lymphoma cells (and isolation thereof), ex vivo cell culture system, candidate therapeutic agent, and components thereof, described herein with respect to the ex vivo culture system and method of identifying an agent that inhibits leukemia or lymphoma also are applicable to those same aspects of the aforementioned method of treating a leukemia or lymphoma.

As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the method described herein comprises administering a “therapeutically effective amount” of a composition comprising the candidate agent. A “therapeutically effective amount” refers to an amount effective, at dosages and for periods of time necessary, to achieve a desired therapeutic result. The therapeutically effective amount may vary according to factors such as the disease state, age, sex, and race of the individual, and the ability of the candidate agent to elicit a desired response in the individual. For example, a therapeutically effective amount of a candidate agent is an amount which decreases the survival and proliferation of leukemia or lymphoma cells.

Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the invention. A composition comprising a therapeutic agent that inhibits leukemia or lymphoma can be administered to a human using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. In some embodiments, the composition is administered to a human using peripheral systemic delivery by intravenous, intraperitoneal, or subcutaneous injection. The composition may be delivered by a single bolus administration, by multiple bolus administrations of the composition, or by continuous infusion administration of the composition.

The ex vivo cell culture system described herein may be used as a model system for drug development and therapeutic assessment for certain types of lymphomas and leukemias. For example, the system may be used as a model for drug development to determine potential efficacy of new compounds prior to clinical trials. In some embodiments, the system may be used by doctors in hospitals to select drugs to treat individual patient tumors (i.e., personalized medicine). In other embodiments, the system may be used by clinical labs to detect minimal residual tumor disease and guide therapy selection to treat and eventually eliminate residual tumors.

The following examples further illustrate the invention but, of course, should not be construed as in any way limiting its scope.

### EXAMPLE 1

This example describes a method of preparing an ex vivo cell culture system comprising bone marrow stromal cells and CLL cells isolated from a patient in which soluble cell signaling molecules are added to the culture medium.

Three sources of bone marrow stromal cells (BMSC) can be used: (1) BM stromal cell line NKTert, (2) BMSC generated from the bone marrow aspirate of a CLL patient (UC CLL021); or (3) CD40L-transduced BMSC. The co-culture system can be performed with 96-well plate, 48-well plate, and 24-well plate depending on the throughput needs.

BMSC may be prepared as follows: (1) Thaw a frozen vial of BMSC and seed into a T-25 ml flask with 6 ml RPMI-1640+10% FBS; (2) Pass the stromal cells when the confluence reaches about 70-80%. Discard the stromal cells if they are over confluent. Usually one flask of 70% confluent stromal cells can be split into 3-4 flasks; (3) Pass every two days for 2-3 times; (4) Prepare single layer of BMSC by (a) trypsinzing the BMSC attached to T-25 flasks and count cell number and (b) resuspending the BMSCs in RPMI-1640+10% FBS and seeding into both 12-well and 24-well plates 24 hours before the co-culture.

2×10⁵ stromal cells may be plated in 2 ml per well onto a 12-well plate and 0.5×10⁵ cells may be plated in 2 ml per well onto a 24-well plate. The seeded 24-well plate will not be used until three days later. One well of a 24-well plate holds 2 ml of medium (ratio of BMSC/tumor cells=1:10 to 1:50).

Primary CLL/lymphoma cells (referred as CLL hereinafter) for co-culture may be prepared as follows:

(1) Make CLL co-culture media: RPMI-1640+20% FBS+penicillin-streptomycin-gentamycin+1:100 insulin-transferrin-selenium supplement;

(2) isolate CLL cells from peripheral blood of patients by either Ficoll (if CLL>90%) or ROSETTESEP™ Human B Cell Enrichment Cocktail kit (Stemcell Cat #15064). Freeze extra CLL cells in 90% FBS and 10% DMSO. Use 5×10⁷ cells for each co-culture assay. This isolation protocol does not apply to MCL tumors;

(3) Pre-incubation: Add 2 ml CLL cell suspension onto the prepared 12-well plate with BMSC monolayer (not 24-well plates). Incubate for 3 days and change media every day. For media change, carefully remove 2 ml of media from 4 ml culture without disturbing the CLL cells/BMSC laid on bottom and add 2 ml pre-warmed CLL co-culture media to the side of the well;

(4) After 3 days of pre-incubation, re-suspend the CLL cells by gently pipetting the co-culture and transfer the cell suspension into a 15 ml tube. Some CLL cells are tightly attached to BMSC such that they cannot be completely removed;

(5) T cell depletion: (a) previous co-culture experience demonstrates that T cells in patient blood proliferate at a much faster pace than B cells or CLL cells and they may take over the co-culture after 7 days for some patients. Thus, the remaining T cells in the co-culture must be removed beforehand, (b) Spin down CLL cells and re-suspend in 1 ml CLL co-culture media in Eppendorf tubes, (c) Anti-CD3-coated DYNABEADS™ (ThermoFisher Scientific, Cat #11151D): wash the beads and add 50 μl into the Eppendorf tube. Rotate tubes at 4° C. for 30 minutes. Place the tube into a magnet for 2 minutes. Use 1 ml pipettes or 3 ml transferring pipettes to transfer the cell suspension into a new Eppendorf tube and load into the magnet again. Repeat the process three times;

(6) Violet CFSE-labeling: (a) Dissolve violet CFSE in 100 μl DMSO and add 10 μl into pre-warmed 10 ml PBS; (b) Spin down cell suspension and re-suspend the cell pellet in 10 ml PBS. Wash the cell pellets one more time with PBS; (c) Re-suspend CLL cell pellets in the PBS with violet-CFSE solution and incubate at 37° C. for 15 minutes; (d) Add 1 ml 100% FBS into the labeled cell suspension, mix well to stop the labeling process and spin down; (e) Re-suspend labeled cells in 10 ml CLL co-culture media.

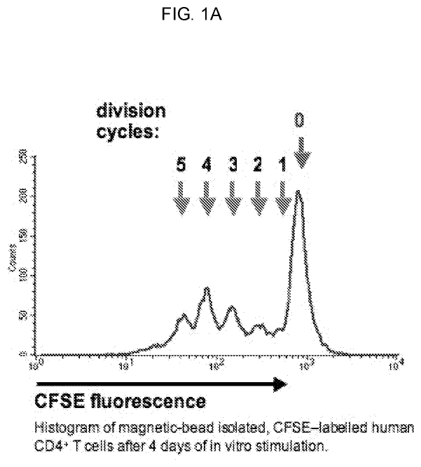

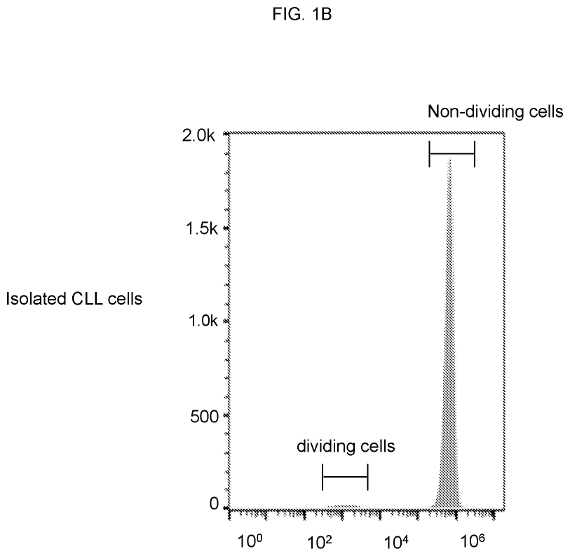

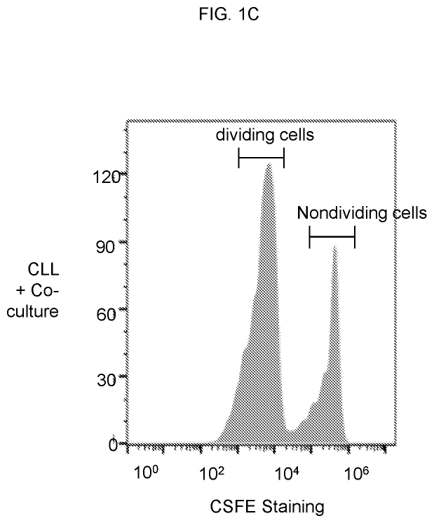

(7) CpG/IL-15 stimulation in 24-well plate with mono-layer BMSC: (a) For each drug sensitivity assay, one well will be left as unstimulated. Take 1 ml of 10 ml labeled CLL cells and add it into the well designated as “unstimulated;” (b) Dissolve CpG and IL-15 powder in co-culture media to make stock solutions. CpG at 1 ug/ul and IL-15 at 10 ug/ul; (c) Add CpG (2 ug/ml final) and IL-15 (10 ng/ml final) into the remaining 9 ml of the labeled CLL cell suspension. Mix well and add 1 ml into the 24-well plate that contains 1 ml of co-culture media (step 5) with 2 ug/ml of CpG and 10 ng/ml of IL-15. The final volume/well will be 2 ml. CLL proliferation may be measured using CFSE-labeling, as shown in FIGS. 1A-1C.

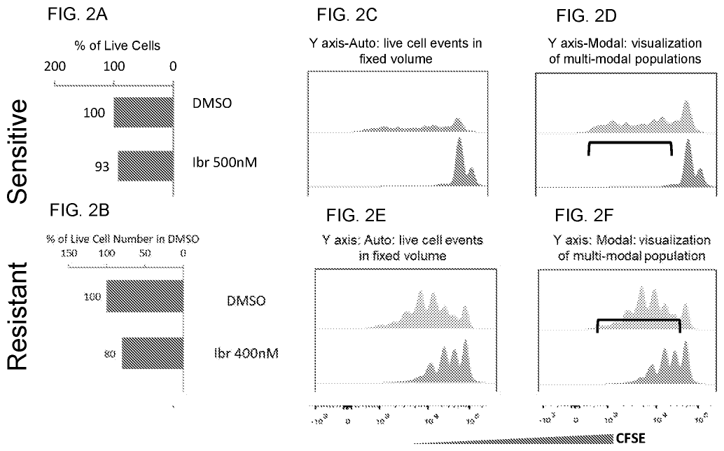

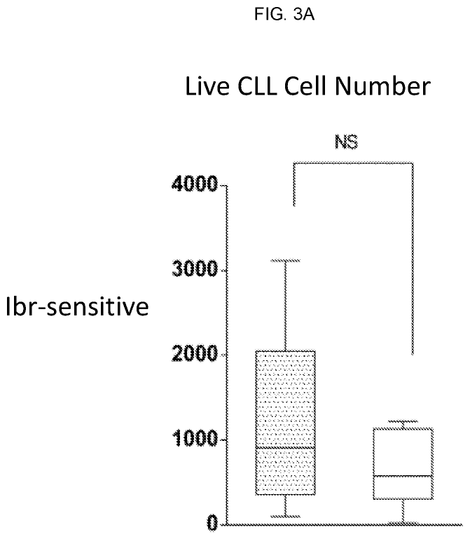

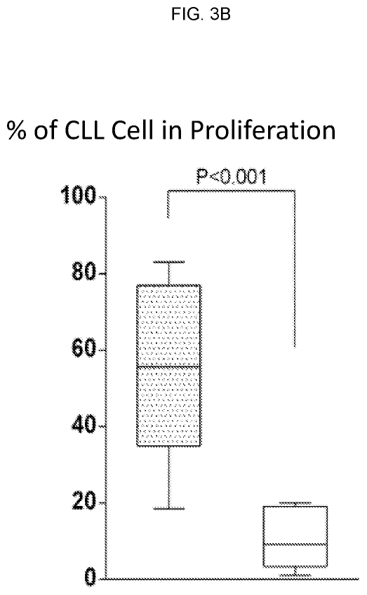

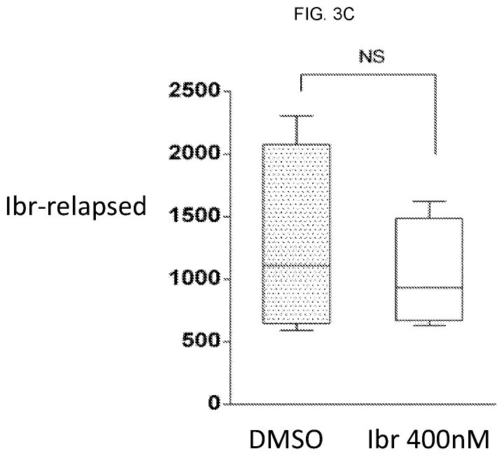

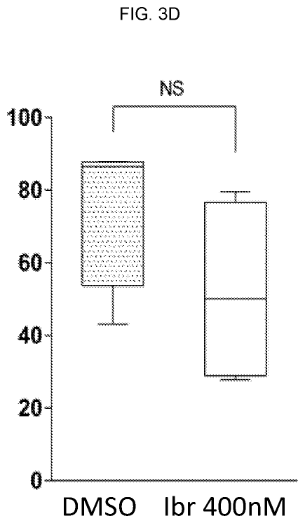

(8) Drug treatment: (a) Drugs will be added the next day. Drugs (Ibrutinib (Ibr), Venetoclax, etc.) are dissolved in DMSO at 1,000× stock solution. Include DMSO control; (b) Add 2 ul per well to the 24-well plate and mix gently; (c) Drug media need not to be changed for the first 2-3 days but must be changed every day afterwards with the following media; (d) Make 10 ml drug solution in co-culture media that contains CpG/IL15. Use co-culture media without CpG/IL15 for “unstimulated” well; (e) Check cell viability and CFSE profile at day 7 of the drug treatment by flow cytometry: gently re-suspend CLL cells and take 100 μl for staining with following antibody/reagents: APC-CD3, FITC-CD19, PI for live cell staining, and channel for BV421 for violet CFSE staining. FIGS. 2 and 3 show results of treating a co-culture of BMSC and CLL cells isolated from a patient with Ibr. Soluble cell signaling molecules (IL-15 and CpG) were added to the co-culture.

### EXAMPLE 2

This example describes a method of preparing an ex vivo cell culture system comprising bone marrow stromal cells (BMSC) and leukemia (e.g., CLL) or lymphoma (e.g., B and T cell lymphomas) isolated from a patient in which the BMSC express one or more cell signaling molecules.

Human CD40L gene (hCD40L) is an important B cell activation molecule and its expression is restricted to activated T cells and dendritic cells. In order to better mimic lymph node (LN) microenvironment, hCD40L was cloned into a lentiviral expression vector and was transduced into a BMSC line. After selection, a stable hCD40L-expressing BMSC cell line was created and referred to as “BMSC-1.”

Interleukin-4 (IL4), Interleukin-15 (IL15), and Interleukin-21 (IL21) are important for optimal B cell activation and are naturally produced by T cells and macrophages. cDNA encoding human IL4, IL15, and IL-21 were cloned into a lentiviral expression vector, which was transduced into BMSC resulting in IL4/IL15/IL21 triple cytokine-expressing BMSCs.

CXCL12 and CXCL13 are ligands for chemokine CXCR4 and CXCR5 and are important for CLL homing, attachment, and activation in the lymph node (LN) microenvironment. The cDNA of human CXCL12/CXCL13 was cloned into a lentiviral expression vector, which was transduced into BMSC resulting in CXCL12/CXCL13-expressing BMSCs. Initial co-culture results showed that CXCL12/CXCL13-expressing BMSC did not promote CLL proliferation. Their effects on tumor cell migration will be determined.

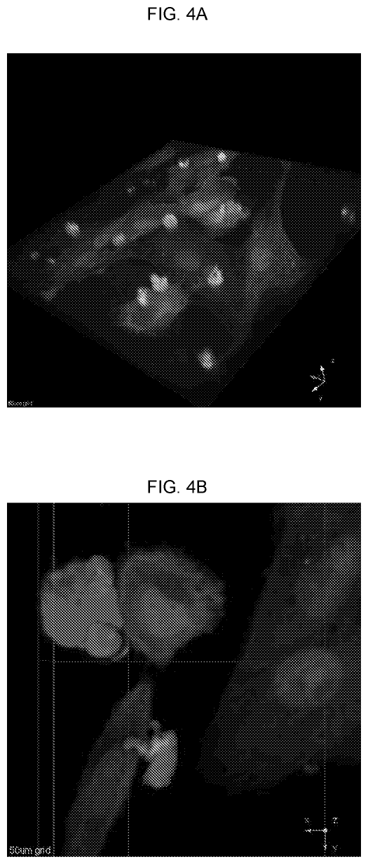

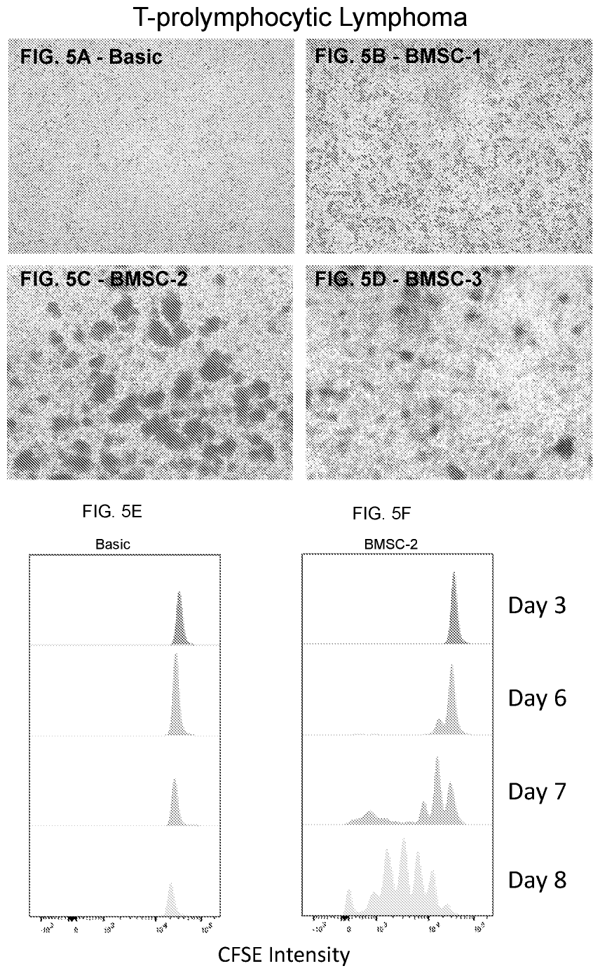

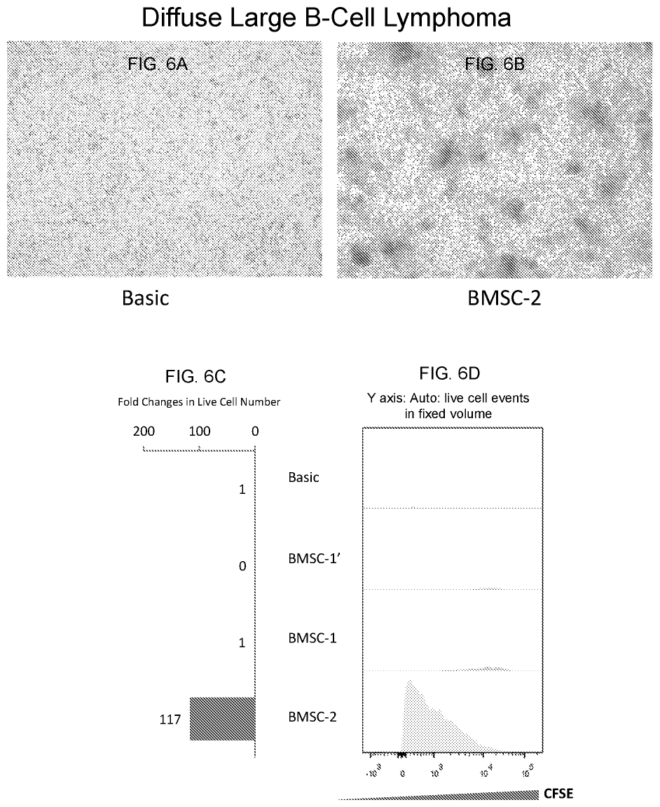

B- and T-cell lymphomas were co-cultured with hCD40L- and triple cytokine-expressing BMSC. In this regard, hCD40L-BMSC and triple cytokine-expressing BMSC were separately cultured in T-25 flasks (or T-75 flasks). The day before the co-culture, BMSC were detached with trypsin digestion and cell number was counted. CD40L-BMSCs and triple cytokine-expressing BMSC were combined at 1:1 ratio and the mixture was plated into 12-well and 24-well plates. The mixture of CD40L-BMSCs and triple cytokine-expressing BMSC was referred to as “BMSC-2.” Primary lymphoma cells, including diffuse large B-cell lymphoma (DLBCL), were isolated and cultured as described in Example 1 to establish a co-culture. Because the BMSC express cell signaling molecules, there was no need to stimulate the co-culture with CpG or IL15. Results of these co-culture experiments are shown in FIGS. 4-8.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the elements described herein, in all possible variations thereof is encompassed by the invention unless otherwise indicated herein or otherwise clearly contradicted by context.

1. An ex vivo cell culture system, which comprises (a) a first cell culture comprising bone marrow stromal cells (BMSC) which express one or more exogenous cell signaling molecules; (b) a second cell culture comprising leukemia or lymphoma cells isolated from a human; and optionally (c) one or more soluble cell signaling molecules.

2. The cell culture system of claim 1, which comprises one or more soluble cell signaling molecules.

3. The cell culture system of claim 1 or claim 2, wherein the one or more exogenous or soluble cell signaling molecules are selected from growth factors, cytokines, chemokines, hormones, a CpG oligodeoxynucleotide, and combinations thereof.

4. The cell culture system of claim 3, wherein the one or more exogenous or soluble cell signaling molecules are selected from an interleukin, a chemokine, a TNF protein superfamily member, and combinations thereof.

5. The cell culture system of any one of claims 1-4, wherein the one or more exogenous or soluble cell signaling molecules are selected from interleukin-4 (IL-4), interleukin-15 (IL-15), interleukin-21 (IL-21), CD40 ligand (CD40L), interleukin-6 (IL-6), interleukin-8 (IL-8), interleukin-10 (IL-10), chemokine (C-X-C motif) ligand 12 (CXCL12), chemokine (C-X-C motif) ligand 13 (CXCL13), chemokine receptors CXCR4 and CXCR5, B-cell activating factor (BAFF), a proliferation inducing ligand (APRIL), and combinations thereof.

6. The cell culture system of any one of claims 1-5, wherein the second cell culture comprises leukemia cells.

7. The cell culture system of any one of claims 1-5, wherein the second cell culture comprises chronic lymphocytic leukemia (CLL) cells, B cell lymphoma cells, or T cell lymphoma cells.

8. A method of preparing the ex vivo cell culture system of any one of claims 1-7, which comprises:
(a) contacting bone marrow stromal cells (BMSC) with a vector comprising at least one nucleic acid sequence encoding at least one exogenous cell signaling molecule;
(b) culturing the BMSC under conditions whereby the at least one nucleic acid sequence is expressed and the at least one exogenous cell signaling molecule is produced;
(c) contacting the BMSC with leukemia or lymphoma cells isolated from a human and optionally one or more soluble cell signaling molecules, and
(d) culturing the BMSC and leukemia or lymphoma cells under suitable conditions whereby the ex vivo culture system is prepared.

9. The method of claim 8, which comprises (c) contacting the BMSC with leukemia or lymphoma cells isolated from a human and one or more soluble cell signaling molecules

10. The method of claim 8 or claim 9, wherein the vector is a viral vector.

11. The method of claim 10, wherein the vector is a lentivirus vector.

12. A method of identifying at least one candidate agent that inhibits leukemia or lymphoma, which method comprises:
(a) treating the ex vivo cell culture system of any one of claims 1-7 with at least one candidate agent;
(b) measuring one or more of survival, proliferation, adhesion, and/or migration of the leukemia or lymphoma cells following step (a), wherein a decrease in survival, proliferation, adhesion, and/or migration of the leukemia or lymphoma cells as compared to cells not contacted with the at least one candidate agent indicates that the at least one candidate agent inhibits leukemia or lymphoma.

13. The method of claim 12, wherein the at least one candidate agent is a small molecule, a chemotherapeutic agent, a biologic agent, or an immunotherapeutic agent.

14. The method of claim 12 or claim 13, wherein the at least one candidate agent decreases survival of the leukemia or lymphoma cells as compared to cells not treated with the at least one candidate agent.

15. The method of any one of claims 12-14, wherein the at least one candidate agent decreases proliferation of the leukemia or lymphoma cells as compared to cells not treated with the at least one candidate agent.

16. The method of any one of claims 12-15, wherein the at least one candidate agent decreases adhesion of the leukemia or lymphoma cells as compared to cells not treated with the at least one candidate agent.

17. The method of any one of claims 12-16, wherein the at least one candidate agent decreases migration of the leukemia or lymphoma cells as compared to cells not treated with the at least one candidate agent.

18. The method of any one of claims 12-17, wherein the survival, proliferation, adhesion, and/or migration of the leukemia or lymphoma cells is subject-specific.

19. A method of treating leukemia or lymphoma in a subject in need thereof, which method comprises:
(a) isolating leukemia or lymphoma cells from the subject,
(b) preparing an ex vivo cell culture system according to the method of any one of claims 8-11;
(c) treating the ex vivo cell culture system with at least one candidate therapeutic agent;
(d) measuring one or more of survival, proliferation, adhesion, and/or migration of the leukemia or lymphoma cells following step (c), wherein a decrease in survival, proliferation, adhesion, and/or migration of the leukemia or lymphoma cells as compared to cells not treated with the at least one candidate therapeutic agent indicates that the at least one candidate therapeutic agent inhibits the leukemia or lymphoma; and
(e) administering the at least one candidate therapeutic agent to the subject, whereby the leukemia or lymphoma is treated.

20. A method of treating leukemia or lymphoma in a subject in need thereof, which method comprises:
(a) isolating leukemia or lymphoma cells from the subject,
(b) contacting an ex vivo cell culture system comprising BMSCs expressing an exogenous signaling molecule with the isolated leukemia or lymphoma cells and optionally one or more soluble cell signaling molecules;
(c) treating the ex vivo cell culture system with at least one candidate therapeutic agent;
(d) measuring one or more of survival, proliferation, adhesion, and/or migration of the leukemia or lymphoma cells following step (c), wherein a decrease in survival, proliferation, adhesion, and/or migration of the leukemia or lymphoma cells as compared to cells not treated with the at least one candidate therapeutic agent indicates that the at least one candidate therapeutic agent inhibits the leukemia or lymphoma; and
(e) administering the at least one candidate therapeutic agent to the subject, whereby the leukemia or lymphoma is treated.

21. A method of treating leukemia or lymphoma in a subject in need thereof, which method comprises:
(a) isolating leukemia or lymphoma cells from the subject,
(b) having the leukemia or lymphoma cells tested for responsiveness to at least one candidate therapeutic agent using a method of any one of claims 12-18; and
(c) administering the at least one candidate therapeutic agent to the subject, whereby the leukemia or lymphoma is treated.

22. The method of any one of claims 19-21, wherein the at least one candidate therapeutic agent is a small molecule, a chemotherapeutic agent, a biologic agent, or an immunotherapeutic agent.

23. The method of any one of claims 12-22, wherein the ex vivo cell culture system is treated with two or more candidate agents.

24. The method of any one of claims 19-23, wherein the subject suffers from chronic lymphocytic leukemia (CLL).

25. The method of any one of claims 19-23, wherein the subject suffers from a non-Hodgkin's lymphoma (NHL) selected from small lymphocytic lymphoma (SLL), follicular lymphoma (FL), marginal zone lymphoma (MZL), mantle cell lymphoma (MCL), diffuse large B-cell lymphoma (DLBCL) or a T-cell lymphoma selected from peripheral T-cell lymphoma and T-prolymphocytic lymphoma.
